(19) United States
(12) Patent Application Publication
(10) Pub. No.: US 2022/0291823 A1
(43) Pub. Date: Sep. 15, 2022

(54) Enhanced Visualization And Playback Of Ultrasound Image Loops Using Identification Of Key Frames Within The Image Loops

(71) Applicant: GE Precision Healthcare LLC, Wauwatosa, WI (US)

(72) Inventors: Arun Kumar Siddanahalli Ninge Gowda, Bangalore (IN); Srinivas Koteshwar Varna, Bangalore (IN)

(21) Appl. No.: 17/198,692

(22) Filed: Mar. 11, 2021

(51) Int. Cl. G06F 3/0485; G06F 16/74; G06F 16/732; G06T 7/00; G06K 9/00

(52) U.S. Cl. CPC G06F 3/04855; G06F 16/743; G06F 16/7335; G06T 7/0012; G06K 9/00744; G06K 9/00765

(57) ABSTRACT

An imaging system and method for operating the system provides summary information about frames within video or cine loop files obtained and stored by the imaging system. During an initial review and analysis of the images constituting the individual frames of the cine loop or video, the frames are classified into various categories based on the information identified in the individual frames, and this information is stored with the video file. When the video file is accessed by a user, this category information is displayed in association with the video file to improve and facilitate navigation to desired frames within the video file. The imaging system also utilizes the category information and a representative image from the video file as an identifier for the stored video file to enable the user to more readily locate and navigate directly to the desired video file.

### BACKGROUND OF THE INVENTION

The invention relates generally to imaging systems, and more particularly to structures and methods of displaying images generated by the imaging systems.

An ultrasound imaging system typically includes an ultrasound probe that is applied to a patient's body and a workstation or device that is operably coupled to the probe. The probe may be controlled by an operator of the system and is configured to transmit and receive ultrasound signals that are processed into an ultrasound image by the workstation or device. The workstation or device may show the ultrasound images through a display device operably connected to the workstation or device.

In many situations the ultrasound images obtained by the imaging system are continuously obtained over time and can be presented on the display in the form of videos/cine loops. The videos or cine loops enable the operator of the imaging device or the reviewer of the images to view the changing and/or movement of the structure(s) being imaged over time. In performing this review, the operator or reviewer can move forward and backward through the video/cine loop to review individual images within the video/cine loop and to identify structures of interest (SOI), that include organs/structures or anomalies or other regions of clinical relevance in the images. The operator can add comments to the individual images regarding observations of the structure shown in the individual images of the video/cine loop, and/or perform other actions such as, but not limited to performing measurements on structures shown in the individual images and/or annotating individual images. The video/cine loop and any measurement, annotations and/or comments on the individual images can be stored for later review and analysis in a suitable electronic storage device and/or location accessible by the individual.

However, when it is desired to review the video/cine loop, in order for an individual to review the individual images containing structures of interest (SOIs) such as anomalous structure/regions of clinical relevance and/or measurements and/or annotations and/or comments on the prior observation of the images, the reviewer must look through each individual image or frame of the video/cine loop in order to arrive at the frame of interest. Any identification of the SOIs like anomalous structure(s)/regions of clinical relevance in the individual images/frames or annotations or measurements or comments associated with the individual images/frames are only displayed in association with the display of the actual image/frame, requiring an image-by-image or frame-by-frame review of the video/cine loop in order to locate the desired frame. This image-by-image or frame-by-frame review of the entire video/cine loop required to find the desired image or frame is very time consuming and prevents effective review of stored video/cine loop files for diagnostic purposes, particularly in conjunction with a review of the video or cine loop during a concurrent diagnostic or interventional procedure being performed on a patient.

In addition, in normal practice a number of different video/cine loop files are stored in the same storage location within the system. Often times, these files can be related to one another, such as in the situation where images obtained during an extended imaging procedure performed on a patient are separated into a number of different stored video files. As these files are normally each identified by information relating to the patient, the date of the procedure during which the images were generated, the physician performing the procedure, or other information that is similar for each stored video file, in order to locate the desired video file for review, the reviewer often has to review multiple video files prior to finding the desired file for review.

Therefore, it is desirable to develop a system and method for the presentation of information regarding the content of an image video or cine loop in a summary manner in association with the stored video/cine loop file. It is also desirable to develop a system and method for the summary presentation of information regarding the individual frames of the video file in which clinically relevant information is located, such as SOIs like anomalies and/or other regions of clinical relevance, to improve navigation to the desired images/frames within the video/cine loop.

### BRIEF DESCRIPTION OF THE DISCLOSURE

In the present disclosure, an imaging system and method for operating the system provides summary information about frames within video or cine loop files obtained and stored by the imaging system. During an initial review and analysis of the images constituting the individual frames of the cine loop or video, the frames are classified into various categories based on the information identified within the individual images. When the cine loop/video file is accessed by user, this category information is displayed in association with the video file. Upon accessing the video file, the category information is presented to the individual along with the video file to identify those portions and/or frames of the video file that correspond to the types of information desired to be viewed by the user to improve navigation to the desired frames within the video file.

According to another aspect of the disclosure, the imaging system also utilizes the category information and a representative image selected from the video file as an identifier for the stored video file to enable the user to more readily locate and navigate directly to the desired video file.

According to another aspect of the disclosure, the imaging system also provides the category information regarding the individual frames of the stored video/cine loop file along with the stored file to enable the user to navigate directly to selected individual images within the video file. The category information is presented as a video playback bar on the screen in conjunction with the video playback. The playback bar is linked to the video file and illustrates the segments of the video file having images or frames classified according to the various categories. Using the video playback bar, the user can select a segment of the video file identified as containing images/frames in a particular category relevant to the review being performed and navigate directly to those desired images/frames in the video file.

According to another aspect of the disclosure, the video playback bar also includes various indications concerning relevant information contained within individual frames of the video file. In the initial review of the video/cine loop, those images/frames identified as containing clinically relevant information are marked with an indication directly identifying the information contained within the particular image/frame. These indications are presented on the video playback bar in association with the video to enable the user to select and navigate directly to the frames containing the identified clinically relevant information.

According to one exemplary aspect of the disclosure, a method for enhancing navigation through stored video files to locate a desired video file containing clinically relevant information includes the steps of categorizing individual frames of a video file into clinically significant frames and clinically insignificant frames, selecting one clinically significant frame from the video file as a representative image for the video file, and displaying the clinically significant frame as identifier for the video file in a video file storage location.

According to another exemplary aspect of the disclosure, a method for enhancing navigation in a video file to review frames containing clinically relevant information includes the steps of categorizing individual frames of a video file into clinically significant frames and clinically insignificant frames, creating a playback bar illustrating areas on the playback bar corresponding to the clinically significant frames and the clinically insignificant frames of the video file and linked to the video file, presenting the playback bar in association with the video file during review of the video file, and selecting an area of the playback bar to navigate to the associated frames of the video file.

According to another exemplary aspect of the disclosure, an imaging system for obtaining image data for creation of a video file for presentation on a display including an imaging probe adapted to obtain image data from an object to be imaged, a processor operably connected to the probe to form a video file from the image data, and a display operably connected to the processor for presenting the video file on the display, wherein the processor is configured to categorize individual frames of a video file into clinically significant frames and clinically insignificant frames, to create a playback bar illustrating bands on the playback bar corresponding to the clinically significant frames and the clinically insignificant frames of the video file and linked to the video file, and to display the playback bar in association with the video file during review of the video file and allow navigation to clinically significant frames and clinically insignificant frames of the video file from the playback bar.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 is a schematic block diagram of an imaging system formed in accordance with an embodiment.

FIG. 2 is a schematic block diagram of an imaging system formed in accordance with an embodiment.

FIG. 3 is a flowchart of a method for operating the imaging system shown of FIG. 1 or FIG. 2 in accordance with an embodiment.

FIG. 4 is a schematic view of a display of an ultrasound video file and indications presented on display screen during playback of the video file in accordance with an embodiment.

FIG. 5 is a schematic view of a display of an ultrasound video file and indications presented on display screen in accordance with an embodiment.

FIG. 6 is a schematic view of a display of an ultrasound video file and indications presented on display screen in accordance with an embodiment.

### DETAILED DESCRIPTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. One or more of the functional blocks (e.g., processors or memories) may be implemented in a single piece of hardware (e.g., a general purpose signal processor or random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Although the various embodiments are described with respect to an ultrasound imaging system, the various embodiments may be utilized with any suitable imaging system, for example, X-ray, computed tomography, single photon emission computed tomography, magnetic resonance imaging, or similar imaging systems.

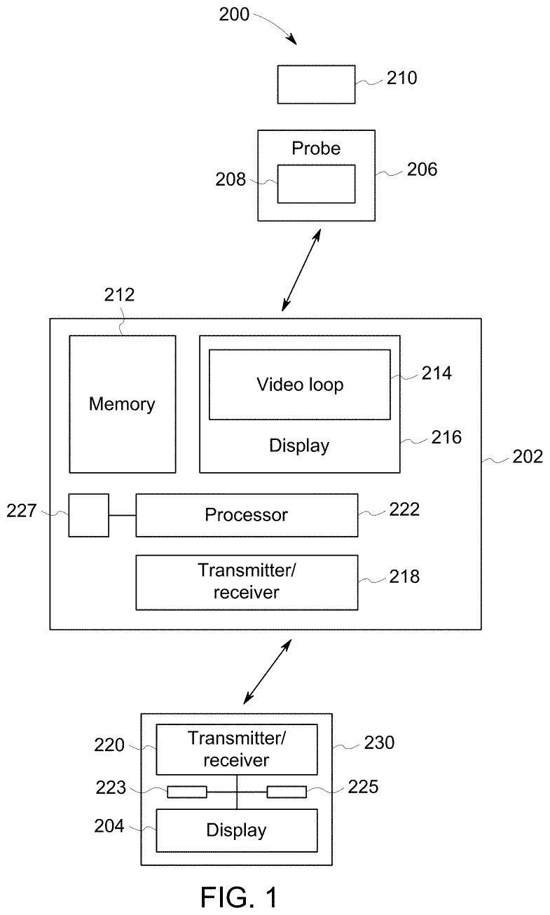

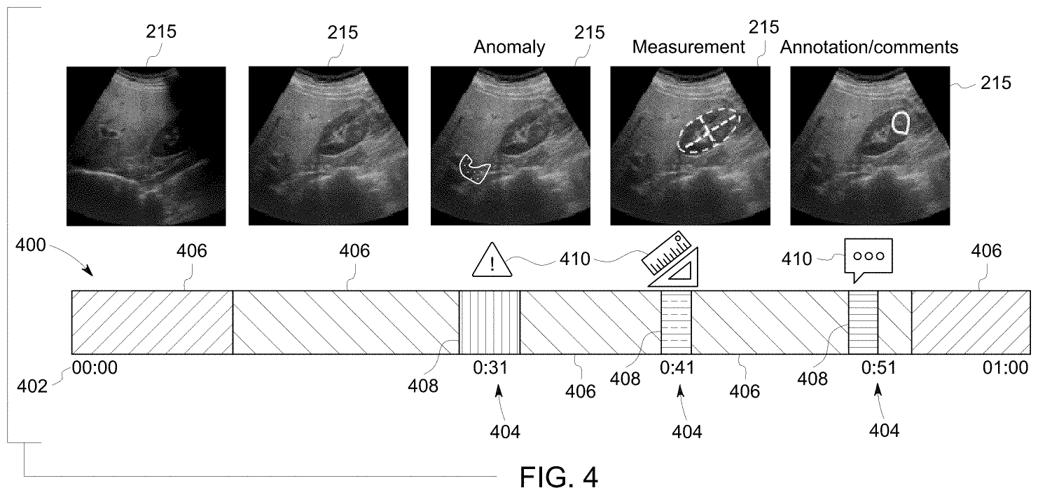

FIG. 1 is a schematic view of an imaging system 200 including an ultrasound imaging system 202 and a remote device 230. The remote device 230 may be a computer, tablet-type device, smartphone or the like. The term “smart phone” as used herein, refers to a portable device that is operable as a mobile phone and includes a computing platform that is configured to support the operation of the mobile phone, a personal digital assistant (PDA), and various other applications. Such other applications may include, for example, a media player, a camera, a global positioning system (GPS), a touchscreen, an internet browser, Wi-Fi, etc. The computing platform or operating system may be, for example, Google Android™, Apple iOS™, Microsoft Windows™, Blackberry™, Linux™, etc. Moreover, the term “tablet-type device” refers to a portable device, such as for example, a Kindle™ or iPad™. The remote device 230 may include a touchscreen display 204 that functions as a user input device and a display. The remote device 230 communicates with the ultrasound imaging system 202 to display a video/cine loop 214 created from images 215 (FIG. 4) formed from image data acquired by the ultrasound imaging system 202 on the display 204. The ultrasound imaging system 202 and remote device 230 also include suitable components for image viewing, manipulation, etc., as well as storage of information relating to the video/cine loop 214.

A probe 206 is in communication with the ultrasound imaging system 202. The probe 206 may be mechanically coupled to the ultrasound imaging system 202. Alternatively, the probe 206 may wirelessly communicate with the imaging system 202. The probe 206 includes transducer elements/an array of transducer elements 208 that emit ultrasound pulses to an object 210 to be scanned, for example an organ of a patient. The ultrasound pulses may be back-scattered from structures within the object 210, such as blood cells or muscular tissue, to produce echoes that return to the transducer elements 208. The transducer elements 208 generate ultrasound image data based on the received echoes. The probe 206 transmits the ultrasound image data to the ultrasound imaging system 202 operating the imaging system 200. The image data of the object 210 acquired using the ultrasound imaging system 202 may be two-dimensional or three-dimensional image data. In another alternative embodiment, the ultrasound imaging system 202 may acquire four-dimensional image data of the object 210.

The ultrasound imaging system 202 includes a memory 212 that stores the ultrasound image data. The memory 212 may be a database, random access memory, or the like. A processor 222 accesses the ultrasound image data from the memory 212. The processor 222 may be a logic based device, such as one or more computer processors or microprocessors. The processor 222 generates an image 215 (FIG. 4) based on the ultrasound image data, optionally in conjunction with instructions from the user received by the processor 222 from a user input 227 operably connected to the processor 222. As the ultrasound imaging system 202 is continuously operated to obtain image data from the probe 206 over a period of time, the processor 222 creates multiple images 215 from the image data, and combines the images/frames 215 into a video/cine loop 214 containing the images/frames 215 displayed consecutively in chronological order according to the order in which the image data forming the images/frames 215 was obtained by the imaging system 202/probe 206.

After formation by the processor 222, the video/cine loop 214 can be presented on a display 216 for review, such as on display screen of a cart-based ultrasound imaging system 202 having an integrated display/monitor 216, or an integrated display/screen 216 of a laptop-based ultrasound imaging system 200, optionally in real time during the procedure or when accessed after completion of the procedure. In one exemplary embodiment, the ultrasound imaging system 202 can present the video/cine loop 214 on the associated display/monitor/screen 216 along with a graphical user interface (GUI) or other displayed user interface. The video/cine loop 214 may be a software based display that is accessible from multiple locations, such as through a web-based browser, local area network, or the like. In such an embodiment, the video/cine loop 214 may be accessible remotely to be displayed on a remote device 230 in the same manner as the video/cine loop 214 is presented on the display/monitor/screen 216.

The ultrasound imaging system 202 also includes a transmitter/receiver 218 that communicates with a transmitter/receiver 220 of the remote device 230. The ultrasound imaging system 202 and the remote device 230 may communicate over a direct wired/wireless peer-to-peer connection, local area network or over an internet connection, such as through a web-based browser, or using any other suitable connection.

An operator may remotely access imaging data/video/cine loops 214 stored on the ultrasound imaging system 202 from the remote device 230. For example, the operator may log onto a virtual desktop or the like provided on the display 204 of the remote device 230. The virtual desktop remotely links to the ultrasound imaging system 202 to access the memory 212 of the ultrasound imaging system 202. Once access to the memory 212 is obtained, such as by using a suitable user input 225 on the remote device 230, the operator may select a stored video/cine loop 214 for review. The ultrasound imaging system 202 transmits the video/cine loop 214 to the processor 232 of the remote device 230 so that the video/cine loop 214 is viewable on the display 204.

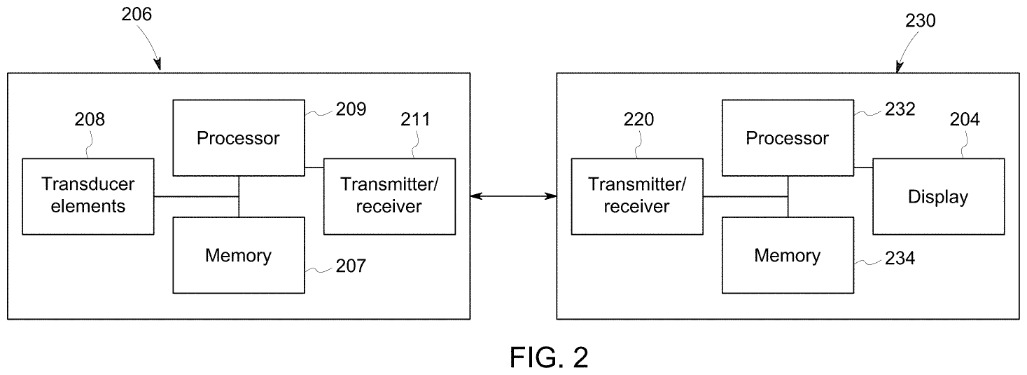

Looking now at FIG. 2, in an alternative embodiment, the imaging system 202 is omitted entirely, with the probe 206 constructed to include memory 207, a processor 209 and transceiver 211 in order to process and send the ultrasound image data directly to the remote device 230 via a wired or wireless connection. The ultrasound image data is stored within memory 234 in the remote device 230 and processed in a suitable manner by a processor 232 operably connected to the memory 234 to create and present the image 214 on the remote display 204.

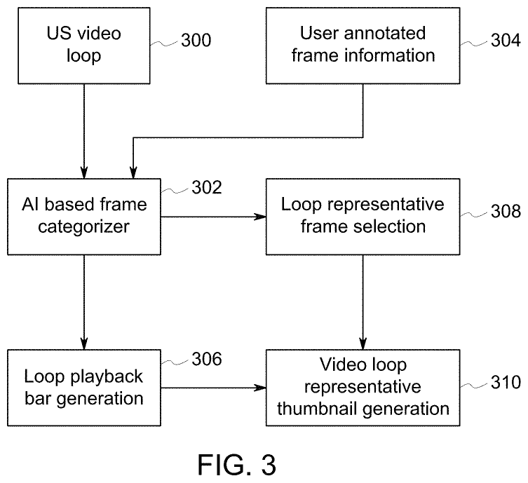

Looking now at FIG. 3, after the creation of the video/cine loop 214 by the processor 222,232, or optionally concurrently with the creation of the video loop 214 by the processor 222,232 upon receiving the image data from the probe 206 in block 300, in block 302 the individual frames 215 forming the video loop 214 are each analyzed and classified into various categories based on the information contained within the particular images. The manner in which the individual frames 215 are analyzed can be performed automatically by the processor 222,232, can be manually performed by the user through the user input 227, or can be performed using a combination of manual and automatic steps, i.e., a semi-automatic process.

According to an exemplary embodiment for an automatic or semiautomatic analysis and categorization of the frames 215, the frame categorization performed in 302 may be accomplished using Artificial Intelligence (AI) based approaches like machine learning (ML) or deep learning (DL), which can automatically categorize the individual frames into various categories. With AI based implementation, the problem of categorizing each of the frame may be formulated as a classification problem. Convolutional neural networks (CNN) a class of DL based networks, which are capable of handling images by design can be used for frame classification achieving very good accuracies. Also recurrent neural networks (RNN) and their variants like long short term memory (LSTM) and gated recurrent units (GRU), which are used with sequential data can also be adapted and combined with CNNs to classify individual frames taking into account the information from the adjacent image frames. ML based approaches like support vector machine, random forest, etc., can be also be used for frame classification, though their performance as well as their adaptability to varying imaging conditions are pretty low when compared to the DL based methods. The models for classification of the frames 215 utilized by the processor 222,232 when using ML or DL can be obtained by training them on the annotated ground truth data which consists of a collection of pairs of image frames and their corresponding annotation labels. Typically, these annotations would be performed by an experienced sonographer wherein each image frame will be annotated with a label that corresponds to its category like good frame of clinical relevance or transition frame or a frame with anomalous structures, etc. Any suitable optimization algorithm, for example gradient descent or root mean square propagation (RMSprop) or adaptive gradient (AdaGrad) or adaptive moment estimation (Adam) or others (normally used with DL based approaches), that minimizes the loss function for classification could further be used to perform the model training with the annotated training data. Once trained, the model can be used to perform inference on new unseen images (image frames not used for model training), thereby classifying each image frame 215 into one of the available categories with which the model was trained on. Further, the classified individual image frames 215 can be grouped into two main categories namely clinically significant frames and clinically insignificant frames. Optionally, if the clinically significant frames 215 contain any structures of interest (SOI) such as organs/structures and/or anomalies and/or other regions of clinical relevance, they can be identified and segmented using a CNN based DL model for image segmentation which is trained on images annotated with ground truth marking for the SOI regions. The results from the image segmentation model could be used to explicitly identify and mark the SOIs within the image frame 215 as well as perform automatic measurements on them.

In the classification process, regardless of the manner in which it is performed, the frames 215 are reviewed by the processor 222,232 determine the nature of the information contained within each frame 215. Using this information, each frame 215 can then be designated by the processor 222,232 into a classification relating to the relevant information contained in the frame 215. While there can be any number and/or types of categories defined for use in classifying the frames 215 forming the video loop 214 by the processor 222,232, some exemplary classifications, such as for identifying clinically significant frames and clinically insignificant frames, are as follows:
a. frames on which measurements were made;
b. frames that provide good, i.e., high quality, images on which to perform a clinical analysis;
c. frames on which there are anomalies associated with the organs/structures in the frames;
d. transition frames (e.g., frames showing movement of the probe between imaging locations)/frames with lesser relevance;
e. frames that a user captured/marked as important/added comments or notes; and/or
f. frames that were captured using certain imaging modes such as, B-mode, M-mode, etc.

By associating each of the frames 215 of the video loop 214 with at least one category, portions 240 of the video loop 214 formed from the categorized frames 215 can be categorized according to the categories of the frames 215 grouped in those portions 240 of the video loop 214, e.g., the clinical importance of the frames 215 constituting each portion 240 of the video loop 214. Also, while certain frames 215 in any portion 240 may have a different classification that others, e.g., a single or small number of frames 215 categorized as transitional are located in a clinically significant or relevant portion of the video loop 214 having mostly high quality images, such as due to inadvertent and/or short term movement of the probe 206 while obtaining the image data, the portions 240 of the video loop 214 can be identified according to the category having the highest percentage for all the frames 215 contained within the portion 240. Additionally, any valid outlier frames 215 of clinical significance or relevance located within a portion 240 containing primarily frames 215 not having any clinical significance or relevance can include indications 408,410 (FIG. 4) concerning those individual frames/images 215.

In block 304, the user additionally reviews the frames 215 in the video loop 214 and provides measurements, annotations or comments regarding some of the frames 215, such as the clinically relevant frames 215 contained in the video loop 214. This review and annotation can be conducted separately from or in conjunction with the categorization in block 302 depending upon the manner in which the categorization of the frames 215 is performed, manual or semi-automatic, or fully automatic. Any measurements, annotations or comments on individual frames 215 are stored in the memory 212,234 in association with the category information for the frame 215.

Using the category information for each frame 215/portion 240 and the measurements, annotations and/or comments added to individual frames 215 from block 302, in block 306 the processor 222 creates or generates a playback bar 400 for the video loop 214. As best shown in FIG. 4, the playback bar 400 provides a graphical representation of the overall video loop 214 that is presented on the display 216,204 in conjunction with the video loop 214 being reviewed, including indications of the various portions 240 of the loop 214, and the frames 215 in the loop 214 having any measurements, annotations or comments stored in conjunction therewith, among other indications.

The playback bar 400 presents an overall duration/timeline 402 for the video loop/file 214 and a specific time stamp 404 for the frame 215 currently being viewed on the display 216,204. The playback bar 400 can also optionally include time stamps 404 for the beginning and end of each portion 240, as well as for the exact time/location on the playback bar 400 for any frames 215 indicated as including measurements, annotations and/or comments stored in conjunction therewith.

The playback bar 400 also visually illustrates the locations and/or durations of the various portions 240 forming the video loop/file 214 on or along the bar 400, such as by indicating the time periods for the individual portions 240 with different color bands 406 on the playback bar 400, with the different colors corresponding to the different category assigned to the frames 215 contained within the areas or portions of the playback bar 400 for the particular band 406. For example, in FIG. 4 the bands 406 corresponding to a portion 204 primarily containing frames 215 identified as not being clinically significant or relevant, e.g., transition frames (e.g., frames showing movement of the probe between imaging locations)/frames with lesser significance or relevance, are indicated with a color different from that used for bands 406 corresponding to a portion 240 primarily containing frames 215 having clinical significance or relevance, such as frames on which measurements were made, frames that provide good, i.e., high quality, images on which to perform a clinical analysis, frames on which there are anomalies associated with the organs/structures in the frames, frames that a user captured/marked as important/added comments or notes, and/or frames that were captured using certain imaging modes such as, B-mode, M-mode, etc.

Further, any individual frame 215 within any of the bands 406 that is identified or categorized as a key individual clinically significant or relevant frame, such as a frame on which measurements were made, a frame on which there are anomalies associated with the organs/structures in the frame, and/or a frame that a user captured/marked as important/added annotations, comments or notes can be additionally identified on the playback bar 400 by a narrow band or stripe 408 positioned at the location or time along the playback bar 240 at which the individual frame 215 is recorded. The stripes 408 can have different identifiers, e.g., colors, corresponding to the types of information associated with and/or contained within the particular frame 215, such that in an exemplary embodiment a stripe 408 identifying a frame 215 containing an anomaly, a stripe 408 identifying a frame 215 containing a measurement, and a stripe 408 identifying a frame 215 containing a note and/or annotation are each represented on the playback bar 400 in different colors. In the situation where adjacent frames 215 are identified as key frames, the stripes 408 representing the adjacent key frames 215 can overlap one another, thereby forming a stripe 408 that is wider than that for a single frame 215. Further, if the key frames 215 are identified the same or differently from one another, i.e., if the adjacent key frames 215 each have an anomaly therein or if one key frame 215 contains an anomaly and the adjacent key frame 215 contains a measurement, the identifiers, e.g., colors, for each key frame can be overlapped or otherwise combined in the wider stripe 408. Similarly, in the case of a key frame 215 having more than one identifier, i.e., the key frame 215 includes an anomaly and a measurement, the identifiers, e.g., colors, associated with the key frame 215 can be combined in the narrow strip 408.

To aid in differentiating these categories and/or types of stripes 408 for individual key images or frames 215 in addition to the differences in the presentation of the respective stripes 408, the playback bar 400 can also include symbols 410 that pictorially represent the information added regarding the particular frame 215. For example, referring to FIG. 4, an individual key clinically relevant frame 215 containing an anomaly, a key frame 215 containing a measurement, and a key frame 215 containing a note and/or annotation can each have a different symbol or icon 410 presented in association/alignment with the location or time for the frame 215 in the playback bar 400 that graphically represents the type of clinically relevant information contained in the particular key frame 215. Further, while the symbols 410 are depicted in the exemplary illustrated embodiment of FIG. 4 as being used in conjunction with the associated stripes 408, the stripes 408 or symbols 410 can be used exclusive of one another in alternative embodiments. Additionally, in the situation where adjacent frames 215 are identified as key frames, forming a stripe 408 that is wider than that for a single frame 215, the stripe 408 can have one or more icons 410 presented therewith depending upon the types of key frames 215 identified as being adjacent to one another and forming the wider stripe 408.

With the playback bar 400 generated using the information on the individual frames 215 forming the video loop/file 214, and with the various aspects 406,408,410 forming the playback bar 400 linked to the corresponding frames 215 of the video loop/file 214 to control the playback of the video loop/file 214 on the display 216,204, the playback bar 400 can be operated by a user via user inputs 225,227 to navigate through the video loop/file 214 to those images 215 corresponding to the desired portion 240 and/or frame 215 of the video loop/file 214 for review. For example, by utilizing the user input 225,227, such as a mouse (not shown) to manipulate a cursor (not shown) illustrated on the display/monitor/screen 216,204 and select a particular band 406 on the playback bar 400 representing a portion 240 of the video loop 214 in a desired category, the user can navigate directly to the frames 215 in that portion 240 indicated as containing images having information related to the desired category. Also, when selecting a stripe 408 or symbol 410 on the playback bar 400, the user will be navigated to the particular frame 215 having the measurement(s), annotation(s) and/or comment(s) identified by the stripe 408 or symbol 410. In this manner, the user can readily navigate the video loop 214 using the playback bar 400 to the desired or key frames 215 containing clinically relevant information by selecting the identification of these frames 215 provided by the bands 406, stripes 408 and/or symbols 410 forming the playback bar 400 and linked directly to the frames 215 forming the video loop 214 displayed in conjunction with the playback bar 400.

Looking now at FIGS. 4-6, after generation of the playback bar 400, optionally using the information generated in the categorization of the frames 215 in block 302, a representative frame 215 for the video loop 214 is selected in block 308 to aid in the identification of the video loop/file 214, such as within an electronic library of video files 214 stored in a suitable electronic memory 212 or other electronic storage location or device. The representative frame 215 is determined from those frames 215 identified as containing clinically relevant information, and is selected to provide a direct view of the nature of the relevant information contained in the video loop 214 containing the frame 215. For example, a frame 215 having a high quality image and containing a view showing an anomaly in the imaged structure of the patient that was the focus of the procedure can be selected to visually represent the information contained within the video loop 214. When the video loop 214 is stored in the memory 212, upon accessing the storage location in the memory 212 where the file for the video loop 214 is stored, the user is presented with a thumbnail image 500 created in block 310 utilizing the selected representative frame 215 to indicate to the user the nature of the information contained in the video loop 214. In this manner, by viewing the thumbnail image 500, the user can quickly ascertain the information contained in the video loop 214 identified by the thumbnail image 500 and determine if the video loop 214 contains relevant information for the user.

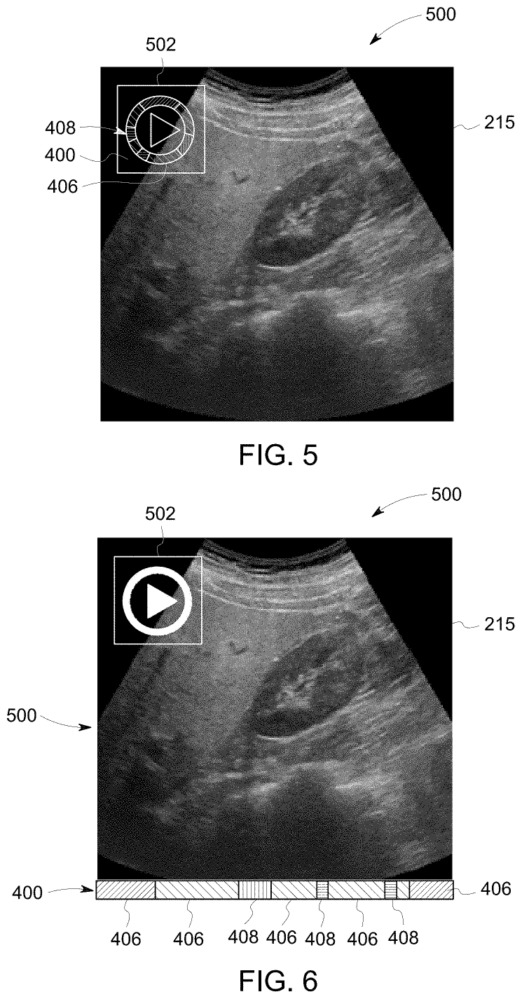

In addition to the representative frame 215, the thumbnail image 500 also additionally presents the user with information regarding the types and locations of information contained in the video loop 214 identified by the thumbnail. As shown in the exemplary embodiment of FIG. 5, the thumbnail image 500 includes a playback icon 502 that can be selected to initiate playback of the video loop 214 on the display 216,204, and in which the playback bar 400 including the bands 406 and stripes 408 is graphically represented in the icon 502. In this manner the user can see the relative portions 240 of the video loop 214 containing clinically relevant information and the general types of the clinically relevant information based on the color of the bands 406 and stripes 408 forming the playback bar 400.

In the exemplary illustrated embodiment of FIG. 6 the thumbnail image 500 includes the playback icon 502, but without the representation of the playback bar 400. Instead, the playback bar 400 is presented directly on the image 500 separate from the icon 502 directly similar to the presentation of the playback bar 400 in conjunction with the video loop 214 when being viewed.

In other alternative embodiments, the summary presentation of the playback bar 400 on the thumbnail image 500 can function as a playback button that is selectable to begin a playback of the associated video loop 214 within the thumbnail image 500. In this manner, the thumbnail image 500 can be directly utilized to show representative information contained in the video loop 214 identified by the thumbnail image 500 without having to frilly open the video file/loop 214.

The written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1-11. (canceled)

12. A method for enhancing navigation through stored video files to locate a desired video file containing clinically relevant information, the method comprising the steps of:
categorizing individual frames of a video file into clinically significant frames and clinically insignificant frames;
selecting one clinically significant frame from the video file as a representative image for the video file; and
displaying the clinically significant frame within an electronic library of video files as an identifier for the video file.

13. The method of claim 12, wherein the identifier is a thumbnail image of the clinically significant frame.

14. The method of claim 12, further comprising the steps of:
creating a playback bar illustrating areas on the playback bar corresponding to the clinically significant frames and the clinically insignificant frames of the video file and linked to the video file; and
displaying the playback bar with the clinically significant frame as the identifier for the video file in the video file storage location.

15. The method of claim 14, wherein the playback bar is a playback icon for initiating playback of the video file.

16. The method of claim 14, wherein the clinically significant frames include a frame on which measurements were made, a frame that provides high quality images, a frame including anomalies, or a frame including user-added information.

17. (canceled)

18. An imaging system for obtaining image data for creation of a video file for presentation on a display, the imaging system comprising:
an imaging probe adapted to obtain image data from an object to be imaged;
a processor operably connected to the probe to form a video file from the image data; and
a display operably connected to the processor for presenting the video file on the display,
wherein the processor is configured to categorize individual frames of a video file into clinically significant frames and clinically insignificant frames, to create a playback bar illustrating bands on the playback bar corresponding to the clinically significant frames and the clinically insignificant frames of the video file and linked to the video file, and to display the playback bar in association with the video file during review of the video file and allow navigation to clinically significant frames and clinically insignificant frames of the video file from the playback bar,
wherein the processor is configured to select one clinically significant frame from the video file as a representative image for the video file and to display the clinically significant frame within an electronic library of video files as an identifier for the video file.

19. The imaging system of claim 18, wherein the processor is configured to display the playback bar with the clinically significant frame as the identifier for the video file in the video file storage location.

20. The imaging system of claim 18, wherein the processor is configured to review the individual frames to locate a key clinically significant frame, wherein the key clinically significant frames include a frame on which measurements were made, frames including anomalies, or a frame including user-added information and to generate a graphical representation of the key clinically significant frame within the playback bar.

21. The imaging system of claim 20, wherein the graphical representation of the key clinically significant frame is a stripe disposed within one of the bands forming the playback bar or a symbol disposed adjacent the playback bar.
